(19) United States
(12) Patent Application Publication
(10) Pub. No.: US 2023/0075879 A1
(43) Pub. Date: Mar. 9, 2023

(54) SYSTEMS AND METHODS FOR QUALITY CONTROL IN DIGITAL PROCESSES

(71) Applicant: Dropworks, Inc., Boulder, CO (US)

(72) Inventors: Matthew Ryan Dunn, Louisville, CO (US); Andrew Carl Larsen, Superior, CO (US); Christopher Michael Perkins, Boulder, CO (US)

(21) Appl. No.: 17/883,512

(22) Filed: Aug. 8, 2022

Related U.S. Application Data
(60) Provisional application No. 63/230,667, filed on Aug. 6, 2021.

(51) Int. Cl. C12Q 1/6848

(52) U.S. Cl. CPC C12Q 1/6848; C12Q 2600/166; C12Q 1/6806

(57) ABSTRACT

Provided herein are systems, methods, and reagents for quality control of digital processes in which a detectable reaction occurs, where an active reference is used in a portion or all of the subsamples used in the process and the active reference is subject to the reaction.

### CROSS REFERENCE

This application claims the benefit of U.S. Provisional Application No. 63/230,667, filed Aug. 6, 2021, which application is incorporated herein by reference.

### BACKGROUND OF THE INVENTION

Digital processes can be useful in a number of different fields. In particular, digital assays have become an important tool for quantifying chemical or biochemical species in scientific research and clinical diagnostics. In a digital assay, a sample is partitioned into a plurality of subsamples; analytical targets to be quantified are distributed amongst the subsamples according to a known probability distribution, in many cases a Poisson distribution. A method is applied to determine whether each of the subsamples comprises the analytical target or targets of interest. Measuring the number of subsamples that did not contain any target as well as the total number of subsamples and comparing to the probability distribution allows for a user to infer the quantity of each analytical target in the original sample. The methods are termed “digital” because only a discrete measurement is taken on each subsample, i.e. whether that subsample contained each analytical target or not. Analytical targets of interest could be atoms, molecules (e.g. DNA or RNA), cells, particulates, viruses, or any other species a user might wish to quantify for scientific, clinical, or other purposes.

It is desirable to be able to determine whether a process such as a reaction, e.g., a process that renders an analytical target detectable, has occurred properly in individual subsamples; it is also desirable to obtain information about individual subsamples to aid in analysis of a sample. Improved methods for these considerations are useful.

### SUMMARY OF THE INVENTION

In one aspect, provided herein are methods.

In certain embodiments, provided herein is a method of quality control in a digital process comprising (i) dividing a sample into a plurality of subsamples, wherein (a) a portion of the subsamples potentially comprise a target of interest; (b) a portion of the subsamples do not comprise the target of interest; (c) each of the subsamples comprises an average of at least one of a first active reference component; (ii) exposing the subsamples to a stimulus that causes a first detectable change in the target of interest, if present, and that also causes a second detectable change in the first active reference component; and (iii) detecting the first change in the target of interest, if present, and the second change in the first active reference component, in individual subsamples. In certain embodiments the plurality of subsamples are in separate containers. In certain embodiments the plurality of subsamples are partitions of dispersed phase comprising the target of interest, if present, and the first active reference component, in a continuous phase. In certain embodiments the partitions are stationary. In certain embodiments the partitions and continuous phase are flowing. In certain embodiments the partitions are polydisperse. In certain embodiments the partitions are monodisperse. In certain embodiments the first detectable change causes a first detectable signal and the second detectable change causes a second detectable signal. In certain embodiments the first and second detectable signals are the same signal. In certain embodiments each of the subsamples comprises an average of at least one of a second active reference component and the method comprises exposing the subsamples to the stimulus to cause a third detectable change in the second active reference component and detecting the third detectable change in the second active reference component. In certain embodiments the first and second active reference components are separate. In certain embodiments the first and second active reference components are separate parts of a single component. In certain embodiments each of the subsamples comprises a passive reference component and detecting the passive reference component in each of the subsamples. In certain embodiments the target of interest and the first active reference component are oligonucleotides, and the stimulus causes a polymerase chain reaction in the target of interest and the first active reference component. In certain embodiments the target of interest and the first and second reference components are oligonucleotides, and the stimulus causes a polymerase chain reaction in the target of interest and in the first and second oligonucleotides. In certain embodiments the first and second reference oligonucleotides use the same pair of primers. In certain embodiments the first and second reference oligonucleotides use the different pairs of primers. In certain embodiments the first and second reference oligonucleotides and their primers have different optimal reaction temperatures.

In certain embodiments provided herein is a method of performing quality control of digital polymerase chain reaction (PCR) in droplets comprising (i) forming an emulsion of a plurality of droplets comprising dispersed phase in a continuous phase, wherein (a) a portion of the plurality of droplets potentially comprises a first oligonucleotide target of interest, (b) a portion of the plurality of droplets does not comprise the first oligonucleotide target of interest, and (c) each of the plurality of droplets comprises a first oligonucleotide active reference component and a second oligonucleotide active reference component, different from the first; (ii) performing PCR on the droplets so that (a) the first oligonucleotide target of interest, if present, is amplified and provides a first detectable target signal, (b) the first oligonucleotide active reference component is amplified and provides a first detectable reference signal, and (c) the second oligonucleotide active reference component is amplified and provides a second detectable reference signal; and (iii) in individual droplets, detecting the first detectable target signal, if present, and detecting the first and second detectable reference signals.

In another aspect, provided herein are compositions.

In certain embodiments, provided herein is a composition comprising a first plurality of subsamples created from a first sample, wherein all or substantially all of the first sample is divided into the plurality of subsamples, and wherein (i) a portion of the subsamples comprise a first target of interest; (ii) a portion of the subsamples does not comprise the first target of interest; and (iii) each of the subsamples comprises a first active reference component. In certain embodiments each of the subsamples further comprises a second active reference component, different from the first active reference component. In certain embodiments each of the subsamples further comprises a passive reference component. In certain embodiments the first target of interest, the first active reference component, and the second active reference component, if present, are oligonucleotides. In certain embodiments each subsample further includes a first target primer set for the first target of interest, a first active reference primer set, different from the first target primer set, for the first active reference, and, if a second active reference component is present, a second active reference primer set for the second active reference. In certain embodiments each subsample comprises a first and second active reference oligonucleotide and first and second active reference primer sets, wherein the first active reference oligonucleotide and the first active reference primer set has a first optimum temperature for amplification and the second active reference oligonucleotide, and the second active reference primer set has a second optimum temperature for amplification, different from the first optimum temperature.

In certain embodiments provided herein is a kit for performing quality control on a digital polymerase chain reaction (dPCR) comprising (i) a first container comprising a first active reference oligonucleotide; (ii) a second container comprising a first set of primers specific for amplifying the first active reference oligonucleotide; (iii) a third container comprising a second active reference oligonucleotide, different from the first active reference nucleotide; and (iv) a fourth container comprising a second set of primers specific for amplifying the second active reference oligonucleotide. In certain embodiments the first and second containers are the same container. In certain embodiments the first and second containers are different containers.

In certain embodiments provided herein is a composition comprising a plurality of droplets of dispersed phase in a continuous phase, wherein all or substantially all of the droplets are formed from a single sample, wherein (i) a portion of the plurality of droplets potentially comprises a first target oligonucleotide; (ii) a portion of the plurality of droplets does not comprise the first target oligonucleotide; and (iii) each of the droplets of the plurality of droplets comprise a first active reference oligonucleotide.

In certain embodiments provided herein is a composition comprising at least two different oligonucleotides and one or more primers for amplifying the oligonucleotides by PCR, wherein the oligonucleotides and the primers comprising modified nucleotides that are not natural and not found in natural samples, and the primers will not amplify natural oligonucleotides.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

Provided herein are systems, methods, and reagents for quality control of digital processes in which a detectable reaction occurs, where an active reference is used in a portion or all of the subsamples used in the process and the active reference is subject to the reaction.

Digital assays have a number of key advantages over traditional means of quantifying the number or concentration of an analytical target or targets in a sample. First, because the method directly counts the number of subsamples unoccupied by an analytical target and compares that to a probability distribution, a direct measurement of the original quantity of an analytical target can be made without reference to a calibration standard. This is especially valuable when construction of such a standard is difficult to do repeatably, accurately, or at all. Second, because only the presence of an analytical target in each subsample must be measured, small or moderate variation or noise in the detection signal does not alter the final determination of presence (and thus quantity/concentration) of the target in the sample. Third, because the volume of each subsample is inherently smaller than the original sample and analytical targets are not typically evenly distributed amongst the subsamples, the relative concentration of the analytical targets in each subsample that contains the targets is higher than in the bulk sample; in cases with many subsamples and low quantities of analytical targets, the increase in concentration can be multiple orders of magnitude. For methods of detecting the presence of analytical targets in subsamples that are sensitive to concentration (e.g. methods mediated by chemical reactions), this can greatly increase the sensitivity of a digital assay for detecting and/or quantifying the analytical target relative to analog alternatives.

Systems and methods provided herein are generally applicable to all digital systems in which subsamples are exposed to a stimulus that produces a detectable change in a target of interest in individual subsamples. For convenience, the systems and methods will be described in detail for digital polymerase chain reaction (dPCR), but it will be appreciated that they are applicable to other digital processes, as appropriate.

In dPCR, the analytical target(s) of interest is a sequence of DNA (or cDNA, if RNA is to be quantified). If the number of molecules of DNA with the sequence(s) of interest is to be quantified in an aqueous sample, PCR reagents, PCR primers specific to the sequence(s) of interest, and an optical probe are added to the aqueous sample. The aqueous sample is then partitioned into thousands, tens of thousands, hundreds of thousands, or even millions of subsamples, in some cases in small physical containers and in other cases in small droplets. If DNA molecules that have the oligonucleotide sequence of interest are in the original sample, these molecules are distributed among the subsamples with a probability approximated by a Poisson distribution. The subsamples are then subjected to PCR thermal cycling and each is optically interrogated to determine whether the sequence of interest amplified in the respective subsample, and the original quantity of DNA molecules with the sequence or sequences of interest can be inferred from the probability distribution. Digital PCR is of great interest to researchers and clinicians for its ability to sensitively detect low numbers of target DNA sequences in biological samples as well as its ability to quantify the target DNA sequences without calibration.

Important to quantifying analytical targets using digital assays is the ability to estimate the uncertainty around the quantification and the reliability of the conclusions made from the measurements. Examples of sources of such uncertainty include sampling error, sampling bias, inefficient or incomplete chemical reactions, low signal strength, low signal-to-noise ratio, or high signal variability, including optical, mechanical, chemical, acoustic, or electrical signals. Improved qualitative and/or quantitative estimation of magnitude of the sources of uncertainty improves evaluation of the accuracy, precision, and reliability of information derived from a digital assay.

One example of a pair of sources of uncertainty in a digital assay is sampling uncertainty and sampling bias. Digital assays involve partitioning a sample into a plurality of subsamples, where one or more assay targets (if present) are distributed among the plurality of subsamples. In order to draw conclusions about the one or more assay targets in the original sample, assumptions must be made about how those targets distribute themselves among the plurality of subsamples. If these assumptions are unfounded or incorrect, the conclusions drawn from the assay may suffer from higher uncertainty or bias the assumptions would indicate. Thus, systems, methods, and reagents for evaluating the validity of the underlying assumptions are valuable to evaluating the validity of conclusions made on analysis of the assay under those assumptions.

A second example of a source of uncertainty in a digital assay is uncertainty around the efficiency of the underlying process, e.g., chemical reaction in each subsample. In order to draw conclusions about one or more assay targets in the original sample, it is assumed that each subsample reacts with similar efficiency. This is especially important in digital assays where the effect of inhibitors is diluted through the partitioning itself, so that inhibitor is not present or present in low amounts in some or most of the subsamples, thus preventing a fractional population of the subsamples from performing. In this case, the uncertainty lies in the inability to distinguish the fractional population of the subsamples that are false or true negatives, affecting the resulting statistical analyses. Thus, systems, methods, and reagents for assessing the efficacy of the underlying chemical reaction would enable evaluation of the validity of the underlying assumptions and conclusions drawn from the resultant data.

In digital PCR, the subsamples are generally either in separate physical containers or are formed as droplets, e.g., stationary droplets or flowing droplets. A typical assumption would be that all of the subsamples have the same volume and that DNA molecules distribute themselves amongst the subsamples according to a Poisson distribution, for which the uncertainty is readily calculated. When the subsamples are in physical containers (e.g. indentations in a silicon chip, microtiter plate wells), partial filling of some of the containers can lead to a bias, where more fully filled containers will be more likely to contain DNA molecules than less fully filled containers. In the case of droplets, if droplets are not monodisperse (i.e., all of the same or substantially the same volume) but are weighted equally in the digital assay analysis, a bias will be introduced into the calculations, as larger volume droplets are more likely to contain DNA molecules than smaller volume droplets. Thus, systems and methods for measuring the volume of individual subsamples, e.g. in individual containers or in droplets, would allow for quantitative assay calculations to be appropriately weighted.

There are many factors that can lead to low signal, low signal-to-noise, or high signal variation in digital assays, including chemical, physical, and electrical system factors. Examples of chemical factors include reagent composition and variability, enzyme and/or target integrity, variability in sample preparation (user error including pipetting error and or sample mixing), target aggregation or colocalization, chemical reaction inhibitors or competitive chemical reagents, or not providing necessary or sufficient conditions for chemical reactions to be complete. Physical examples include detector alignment, partition position, partition volume and shape, detector hardware, detector response, temperature, vibrations, and the like.

In digital assays, low/poor signal or incomplete assay reactions can create issues with data interpretation, as it can become difficult to distinguish between cases where no targets were present in partitions and those in which conditions were not sufficient to generate a signal from subsamples in which targets were actually present. For example, in a digital PCR assay it can be difficult to distinguish between samples that contain no target DNA and those in which some component of the analytical system (e.g. the thermal cycler) failed to amplify the target DNA or read a signal from the target DNA. Additionally, in droplet-based systems, if there is little or no signal from a subsample droplet, the presence of the droplet itself may be difficult to detect. An accurate count of the number of droplets without the analytical target is necessary to determine the starting quantity of the analytical target in the bulk sample. Systems and methods for identifying cases where conditions were not sufficient to generate an assay signal from partitions or where signal was too low to distinguish between truly empty partitions and occupied partitions are needed to improve the quality of such digital assays.

In order to perform quantitative analysis of digital assays, both the number of occupied (i.e., containing at least one copy of a target of interest) and unoccupied (i.e., not containing the target of interest) subsamples must be known. If the systems or methods employed to count unoccupied subsample rely on a signal being measured from those subsamples, a signal that is too low (either in absolute value or relative to system noise) may result in undercounting the total number of unoccupied subsamples. For example, in droplet-based digital PCR, droplets are generated containing either probes that are acted upon by polymerase enzymes such as unhydrolyzed hydrolysis probe species or probes that interact with the final DNA targets in such a way to produce increased fluorescence, such as intercalating agents or molecular beacons. For hydrolysis probes, in the absence of a PCR reaction for a target specific to the hydrolysis probe species, fluorescence from the probe is largely quenched and the droplet remains dark as it is interrogated by the detection element. If the dark droplets are not recognizable and countable by the detection element and underlying signal processing algorithm, then those droplets will not be counted and affect the resultant statistical calculation of molecule count and/or volumetric concentration.

Thus, there is a need for systems and methods to perform quality control measurements on digital assays to assess variation, e.g., polydispersity of subsample volume, the efficiency of the underlying chemical reaction in each subsample, the success of the analytical system for detecting subsamples that contain analytical targets of interest, and/or detection of the presence of subsamples that do not contain analytical targets of interest. Provided herein are systems and methods for accomplishing these objectives, in some cases simultaneously accomplishing all of the objectives.

Provided herein are systems and methods for performing measurements in digital assays that allow for measurement, analysis, evaluation, and control of the quality of other measurements made by digital assays. As used herein, “quality” includes a quantitative or qualitative assessment of the accuracy, precision, repeatability, reproducibility, sensitivity, and/or specificity of the digital assay, or any other property of the assay related to the confidence in which a user may make conclusions or decisions based on the results for one or more quantitative or qualitative measures made by the digital assay.

Systems and methods provided herein provide one or more active reference components, i.e., components that are not passive to the reaction being analyzed but are activated through the same mechanism utilized to analyze the target of interest. The one or more active reference components can be present in sufficient concentration to occupy every subsample in the plurality to ensure that each subsample in the plurality is identified and analyzed. The active reference components differ from passive reference components, i.e., components that are present and detectable in the subsamples but that are not subject to the process occurring in the subsamples, e.g., passive reference dyes, in their ability to distinguish the efficacy of the reagents utilized to assay the target of interest. In certain embodiments, a passive reference component is used in addition to one or more active reference components.

Digital assays in which a sample is partitioned into many subsamples each of which is interrogated, and the resultant data is converted into a molecular count and/or volumetric concentration, are dependent on the ability to split the sample into distinct and separate partitions. There are many methods for performing the partitioning including partitioning into physical structures such as wells or compartments that are designed to hold specified volumes of sample or partitioning through the use of immiscible continuous phase, e.g., oils, to stabilize subsample partitions in the form droplets of dispersed phase, e.g., aqueous fluid in an immiscible continuous phase. The droplets may be stationary or flowing, or a combination thereof. In certain embodiments continuous phase comprises an aqueous fluid and dispersed phase comprises an oil.

In embodiments of dPCR in which subsamples are provided in separate containers, or as stationary droplets, in general, after reaction has occurred, only the presence and magnitude of a signal related to one or more reaction products may be detected. dPCR systems that utilize flowing droplets allow more nuanced data to be collected from individual droplets. dPCR systems utilizing flowing droplets may detect the presence or absence of analytical targets of interest in the droplets individually as the pass through a detection system. A signal collected continuously by an element of a detection system may be analyzed with respect to time. This data typically may resemble pulses or peaks due to signal rising from baseline as each droplet enters the detection system, peaking to a maximum value as the droplet passes through the detection system, and then falling back to baseline as the droplet leaves the detection region. The major characteristics (such as height, width, shape) of these peaks have utility in the interpretation of the contents and quality of the droplets being analyzed.

A droplet signal provided by the height of a peak may be indicative of the amount of reaction product produced within that droplet. In the context of digital PCR, signal intensity from the droplet is correlated to amplification of a nucleic acid sequence of interest. If no starting template is present in the droplet, the signal will remain very low. The presence of one or more starting template molecules may result in significantly more signal after the droplet is reacted. As the signal intensity increases, the height of a data peak increases as the droplet passes the detector. In this context, positive droplets can be differentiated from negative droplets based on the characteristic signal change at the detector and the count of positive and negative droplets for each signal can be converted into a starting concentration of DNA within the sample being analyzed.

The application of droplet-based analysis can be challenged by deviations in the size of droplets since the calculated concentration from the droplet plurality assumes the droplets are of a fixed, invariable size and therefore a known volume. Therefore, droplet width may be a useful statistic to either (1) remove faulty/broken/improperly sized droplets from the plurality to not skew the statistical analysis or (2) to provide an actual measure of droplet volume rather than using an assumption. An actual droplet volume can be calculated assuming the droplets move through the detection region at a constant velocity and both the sampling rate of the detector and the geometry of the conduit and/or channel the droplets are passing through are known.

For example, a common error mode in droplet-based analysis is the fusion of two or more independent droplets in the droplet plurality into one larger volume droplet. Due to the increased volume of the droplet, its peak width (or the number of data points per droplet) may present itself as an outlier from the rest of the plurality. This may manifest in the data in many ways including (1) a peak width larger than the rest of the plurality as a result of its larger volume it takes longer for the entirety of the droplet to pass through the detection region, (2) many peak widths smaller than the rest of the plurality as a result of the larger volume droplet being split into many smaller droplets in a device used to increase the spacing of the droplets before the detection region.

The removal of these error mode improves quantification accuracy reducing the probability of false negatives and positives.

Reference signals included in the droplets, e.g. from an active reference component, may be detected in one or more of the detection channels in the detection system. In certain embodiments, a reference signal can be detected using the same detection channel as one or more targets of interest. In certain embodiments, a reference signal can be detected using a separate, additional channel devoted specifically to the detection of the reference signal. Reference signals can arise from passive reference dyes or active reference components that require the partitions to first react, either in their stationary location, e.g., separate containers for partitions or stationary droplets, or in a moving system, e.g., in a flowing PCR system, droplets first pass through a reactor that changes the temperature of the droplet before the signal will appear.

In addition to droplet quality determination, reference signals ensure that each droplet of the plurality is detected regardless of whether the droplet is positive for the detection of target analyte. The reference signal may or may not affect the dynamic range of the detection system itself. For example, if a reaction is loaded with 1 unit of reference dye, and the total signal possibly generated from the reaction is equivalent to 10 units of passive reference dye, the detection window is limited to reaction ranges between 1 and 11 units for dynamic range of 11 units. Lowered starting amounts of reference dye increases both dynamic range and sensitivity to low amounts of signal generated by the reaction within the droplet.

The reference dye typically needs to be a substance that does not interfere in the specific detection of the target of interest during droplet analysis.

While useful in determining droplet quality and in calculating statistics on the droplet plurality, passive references dyes are limited to providing information on the droplet characteristics. It would be of utility to have an active reference dye system that reports not only on droplet quality as well as the quality of the reaction reagents themselves.

In the context of digital PCR, this active reference dye system may take on many forms including but not limited to a control assay in a separate control channel that allows quality information on both the reaction efficiency as well as droplets.

For example, the failure of a reaction to produce detectable signal is often obscured with the possibility that the underlying test is not functional. Therefore, a positive control reaction is accompanied in the sample set to validate a negative result. Certain systems and compositions provided herein embed positive control reactions into each droplet within the plurality providing both functional feedback on the analytical mechanism utilized to analyze the target of interest as well as statistics about the droplets themselves.

The described methods may be utilized in the analysis of any molecule type in digital analysis including but not limited to protein concentration, enzyme function, nucleic acid quantification, and/or small molecule quantification. Typically some form of stimulus is utilized to initiate and/or propagate a reaction being analyzed. This may include thermal energy, electromagnetic energy, chemical additives, and the like.

Systems and methods provided herein utilize production of subsamples in a digital system, where each of the subsamples contains at least one copy of at least one active reference component, and a portion of the subsamples potentially contain a target of interest. In certain embodiments, each of the subsamples contains at least one copy each of two different active reference components. As used herein, a “target of interest” includes a material that is potentially present in the sample from which subsamples are derived, and that, if present, is partitioned into at least a portion of the subsamples when they are formed and is subject to a reaction in the system that causes formation of a reaction product of the target of interest that can produce a detectable signal. For example, in a dPCR system, a target of interest can be a nucleic acid with a specific sequence that can be amplified by PCR, where the amplification products are detectable by use of a one or more optical probes, e.g., fluorescent dyes. As used herein, an “active reference component,” also referred to herein as an “active reference material,” includes a component that is subject to the same reaction or reactions as a potential target in the subsamples, where the reaction causes formation of a product of the active reference component that can produce a detectable signal, which can be different from or the same as the detectable signal from a reaction product of a target of interest, and where the reaction of the active reference component is independent of, or substantially independent of a reaction of a target of interest, if present, or the reaction of the active reference component is related to a reaction of a target of interest, if present, in a known manner. For example, in dPCR systems, an active reference component can be an oligonucleotide that is subject to amplification by PCR, where the amplification products are detectable by use of one or more optical probes, e.g., fluorescent dyes. In certain embodiments, subsamples can also comprise at least one copy of a passive reference component. As used herein, a “passive reference component,” also referred to herein as a “passive reference material” includes a material that is not subject, or not substantially subject, to the same reaction as a potential target of interest but that provides a detectable signal in the subsamples. For example, in a dPCR system a passive reference component can be a dye, e.g., fluorescent dye, that is not subject to PCR reactions. In certain embodiments, the digital process is a digital PCR process. In certain embodiments, the dPCR process is a process wherein subsamples are droplets and the droplets flow through an area where they are influenced by a reactor that causes amplification of the one or more active reference components to produce amplified active reference component products that produce a first detectable signal and one or more targets of interest, if present, to produce amplified target of interest that produces a second detectable signal, and, optionally, a passive reference component, and then through a detector configured to detect one or more signals from the reaction products of the one or more active reference components, reaction products of the one or more targes of interest, if present, and, optionally, the passive reference component. In certain embodiments, the droplets flow through an interrogation region in a conduit in the detector; in certain of these embodiments, the cross-sectional area of the interrogation region is less than the average spherical diameter of the droplets so that the droplets become elongated when flowing through the detector. In certain embodiments, the presence, absence, or magnitude of one or more signals from the detector from the one or more reaction products of active reference components are used to determine one or more characteristics of individual subsamples, such as subsample count, e.g., droplet count, subsample acceptance or rejection, statistical correction of concentration determined in subsample, e.g., droplet analysis, and/or subsample size, e.g., droplet size and volumetric determination, e.g., for accurate quantification of sample volume prior to emulsification. For example, presence, absence, or magnitude of a signals from the detector from the one or more active reference components in a subsample can be used to determine total number of subsamples to be counted. For example, in a dPCR system, droplets in which a signal, or a signal over a threshold value, from the active reference component is detected, are counted and droplets from which a signal is not detected or not over the threshold are not counted. Droplets that are counted that also have a signal from a target of interest, or a signal over a certain threshold, are considered positive for the target of interest, and droplets that are counted that do not have a signal from the target of interest, or a signal over a certain threshold, are considered negative for the target of interest. Thus, systems and methods provided herein allow a user to distinguish between subsamples in which the desired reaction has occurred and those where the desired reaction has not occurred or has not occurred to a sufficient degree. In addition, in certain embodiments systems and methods provided herein allow a user to determine one or more characteristics of individual subsamples, such as individual droplets, such as subsample volume, e.g., droplet volume. In these embodiments, it is possible to base calculations of concentration of target of interest on actual volumes of each partition, rather than relying on assumed volumes; thus, though a monodisperse set of partitions is desirable it is not necessary for calculations. In certain embodiments, a monodisperse partition formation is assumed, along with number of partitions formed, to provide final volume and concentration.

In certain embodiments, a plurality of subsamples is provided, where the subsamples are subsamples of a parent sample, and comprise all or substantially all of the subsamples formed from the parent sample, and where each of the subsamples contains at least one of a first active reference component. In certain embodiments, each of the subsamples also contains at least one of a second active reference component, which can be provided as a material, e.g., an oligonucleotide in PCR systems, that is the same as or different from a material, e.g., an oligonucleotide, providing the first active reference component. A portion of the subsamples can comprise a first target of interest. Each of the plurality of subsamples can also contain a passive reference component. Each of the plurality of subsamples can also comprise other components and/or reagents necessary or useful in a reaction to which the subsamples have been or are to be subjected. In PCR systems, this can include a first set of target primers specific for the first target of interest (nucleic acid), and a first set of reference primers specific for the first active reference component (oligonucleotide), and, if a second active reference component is present, a second set of reference primers that is specific for the second active reference component (oligonucleotide); the first and second reference primers can be the same or different, as described more fully below. Also included are one or more detectable probes that are specific for amplification products of the first active reference component, amplification products of the first target of interest, if present, and amplification products of the second active reference component, if used. The detectable probes for the amplification products of the first active reference component and amplification products of the first target of interest can be the same or different, as described more fully below. The subsamples also contain reagents necessary or useful for performing PCR, as are known in the art.

In certain embodiments, provided is a method comprising providing a sample potentially containing a first target of interest and a first active reference component, and dividing the sample into a plurality of subsamples, each of which contains at least one copy of the first active reference component, and a portion of which contains a copy of the first target of interest; typically, a portion of which do not contain a copy of the first target of interest. Further components present in the sample and subsamples can include any necessary components for reaction of the first target of interest, if present, and reaction of the first active reference component. For example, in dPCR systems, the sample and each subsample can further contain one or more sets of primers to amplify the first active reference component (oligonucleotide), the target of interest (nucleic acid), if present, and reagents necessary or useful in performing PCR on components of the subsamples. Further potential components are as outlined in the previous paragraph and include, e.g., a second active reference component present in the sample and present in each of the subsamples, and/or a passive reference component, present in the sample and each subsample. Other components can include those necessary for reaction of the second active reference component, e.g., in a PCR process, primers for the second active reference. The sample can be provided as a sample in which all necessary components are present, where the sample is, e.g., contained in a sample container, and the sample can be transported from the sample container by an intake system to a process system via an intermediate component, e.g., an injector. In certain embodiments, the intermediate component is configured so that the intake system and the process system are never in continuous fluid communication. The process system can comprise a partitioner for dividing the sample taken up by the process system into a plurality of subsamples, e.g., partitions, and a reactor, for subjecting subsamples, e.g., partitions to a stimulus, such as a chemical or physical stimulus, to initiate and/or promote a reaction in the subsamples. In certain embodiments the reactor is a thermal cycler for PCR. The process initiated and/or promoted by the reactor is a process that causes a first detectable change in the first target of interest, if present, and a second detectable change in the first active reference component. Thus, all subsamples in which a reaction has occurred are detectable by the second detectable change. The reaction of the first target of interest and the first active reference component can be independent or substantially independent of each other; for example, in a dPCR system, the first active reference oligonucleotide and/or its primers can be modified, as described herein, so that they do not cross-react, or do not substantially cross-react, in normal PCR reactions, e.g., in reactions to which the first target oligonucleotide is subject. Thus the degree of detectable change in the first target of interest, for example, intensity of a first signal from a first probe that interacts with reaction products of the first target of interest, can be independent of, or substantially independent of, the degree of detectable change in the first active reference component; for example, intensity of a second signal from a second probe that interacts with reaction products of the first reference component. The first and second signals can be the same or different; in the former case, intensity of the signal indicates whether just the first active reference component is present or both active reference component and first target of interest are present. For example, in a dPCR system the amplification of the first active component and the first target of interest may be detected by signals from a first and second probe, respectively, where the first and second probes can be the same or different. Subsamples in which a signal from the first active reference are not detected can be discarded and subsequent analysis can be adjusted accordingly. Adjustments can include but not be limited to subsample count, acceptance/rejection, statistical correction of concentration determined in subsample analysis, and in some cases, subsample size and volumetric determination for accurate quantification of sample volume prior to subsample formation. For example, in a dPCR system in which the subsamples are partitions (droplets) of a dispersed phase in a continuous phase, adjustments can include but not be limited to droplet count, acceptance/rejection, statistical correction of concentration determined in droplet analysis, and in some cases, droplet size and volumetric determination for accurate quantification of sample volume prior to emulsification. Concentration of the first target of interest can be determined by any number of signals other than the signal from the first active reference component. For example, in a droplet-based system, a concentration of the first target of interest can be obtained based on the number of target positive droplets and negative droplets. The statistics may be corrected by any suitable method, for example by applying a Poisson distribution.

The sample and/or subsequent subsamples can further comprise additional components. In certain embodiments, each subsample comprises a second active reference component, different from the first active reference component, and the process initiated and/or promoted by the reactor is a process that causes a third detectable change in the second active reference component. The first and second active reference components can be separate or can be different parts of a single component. In a dPCR system, the second active reference component can be a second oligonucleotide, different from the first oligonucleotide of the first active reference. Primers for the two active references can be the same or different. The first and second active reference oligonucleotides can be separate oligonucleotides or can be different parts of a single oligonucleotide. In certain instances related to nucleic acid quantification, the reference material comprises one primer set that amplifies two physically distinct and sequence distinct oligonucleotide sequences. Thus, embodiments include a first active reference nucleotide and a first primer set specific to the first active reference oligonucleotide. In certain embodiments, a second active reference nucleotide is included, different from the first, and a second primer set specific for the second active reference nucleotide. The second active reference oligonucleotide may be a separate oligonucleotide from the first active reference oligonucleotide or a different section of the same oligonucleotide. The first and second primer pairs may be the same or different. In certain cases, the first and second primer pairs are the same and are degenerate so that both the first and second active reference oligonucleotides are amplified. E.g., the primer sets may comprise nucleic acid modifications to provide binding degeneracy allowing the primer sets to bind to a number of specifically determined sequence targets allowing multiple reference site to be amplified with a single primer pair. Either sequence-specific detection probe or a sequence independent detection dye may be utilized for reference visualization in the droplets. The second reference oligonucleotide and/or its primers may be modified so that its reactions don't overlap, or do not substantially overlap, with reactions of the first target nucleic acid. The first and second active reference components may have different optimum reaction conditions; for example, in a dPCR system, the first and second oligonucleotides and/or their respective primers can have different optimum reaction temperatures, as described elsewhere herein. In certain instances the primer set may comprise any number or composition of modifications to enable binding specificity to only the target sequence as well as provide the thermal stability to bind at any temperature set point utilized during the analysis. In certain instances, the primer sets may comprise nucleic acid modifications to provide binding degeneracy allowing the primer sets to bind to a number of specifically determined sequence targets allowing multiple active reference components (separate or different parts of a single component) to be amplified with a single primer pair. In any of these instances, the reaction may proceed through a typical thermal cycling reaction in order to exponentially amplify signal similarly to the nucleic acid of interest being analyzed. The above mentioned instance may include the activation of more than one primer set at any given temperature set point. For example, primer sets designed to bind a high temperature may also bind specifically at low temperatures while primer sets designed to bind a low temperature are unable to do so at high temperatures. This may indirectly give a readout to the actual temperature experienced during the reaction in case there is an error in set point and readout in the instrument performing the droplet analysis. In any of these instances, the nucleic acid modifications include but are not limited to locked nucleic acid, peptide nucleic acid, and the like.

In certain embodiment, all subsamples also may provide a passive reference signal, i.e., one or more signals that are present regardless of whether a reaction has taken place or not. In some cases, the signal or signals is provided by components of the subsamples that are already present in the subsamples, e.g., from continuous phase, dispersed phase, components within one or both phases that remain during and after reaction, or with no reaction, and the like. In some cases, a passive reference component is added. For example, in a dPCR system where droplets pass through a detector, the presence or absence, peak height, peak width, and the like, of a droplet may be indicated by components already present in each droplet, or a detectable dye may be present in each droplet that provides a signal regardless of whether or not a reaction has occurred. As indicated elsewhere, in some cases an active reference component may perform the same function, at least in droplets in which reaction has occurred.

The process system can further comprise a detector for detecting one or more detectable characteristics of the subsamples. In certain embodiments, the subsamples are droplets, e.g., droplets of dispersed phase from the sample in a continuous phase, wherein the dispersed phase and continuous phase are substantially immiscible, e.g., wherein the dispersed phase is aqueous and the continuous phase is an oil, e.g., a fluorinated oil. In certain embodiments, subsample droplets flow in a conduit comprising an interrogation region through the detector, wherein the cross-sectional area of the interrogation region is less than the average cross-sectional area of the droplets.

The presence of at least a first active reference component in each droplet allows for several different features.

First, subsamples, e.g., droplets, in which a desired reaction has occurred, e.g., nucleic acid amplification, can be distinguished from those where reaction has not occurred, and/or has occurred to a limited extent. In the latter cases, the subsamples can be discarded for analysis as it is not known whether or not they contained a target of interest. The proportion of subsamples thus discarded can provide useful information. For example, if the proportion of subsamples discarded is high enough, the entire run may be discarded. In addition, if the presence of one or more inhibitors of the desired reaction, e.g., nucleic acid amplification, is suspected, the inhibitor or inhibitors may be present in only a portion of the subsamples, and/or present in different concentrations in different subsamples. A user can determine, e.g., from sample to sample whether inhibitor concentration is changing by analyzing the proportion of subsamples discarded. In addition, the performance of the desired reaction can also be evaluated and, if necessary, other measurements can be normalized if the performance is changing as subsamples are reacted. For example, if the active reference component shows a change in signal from subsample to subsample, corresponding signals from, e.g., a target of interest, may be normalized according to the change. Finally, in some cases it can be assumed that droplets in which the active reference did not react nonetheless have the same likelihood of containing the target of interest as those droplets in which the active reference did react; and in some cases it can be assumed that unreacted droplets also have the same volume distribution as reacted droplets. In certain cases, using these assumptions, the total count droplets containing target of interest can be adjusted, as well as volume of sample.

In addition, since the signal from reaction products of at least one active reference component will generally be present in all subsamples evaluated (not including any subsamples discarded because insufficient reaction occurred), it can also be used as a measure of subsample characteristics; that is, it acts in some ways similar to a passive reference component, providing a detectable signal in every subsample, or at least in every subsample to be evaluated. The simplest characteristic is simply droplet count, where each droplet in which signal from reaction products of at least one active reference component is counted as part of the total. Other characteristics can be particularly useful in droplets moving through an interrogation region in a detector, as described herein. Such characteristics can be useful in, e.g., evaluating droplet volume: if flow rate through the interrogation region is known and time that the droplet is in the interrogation region can be determined by the time of appearance and disappearance of a signal from the droplet provided by at least one active reference component, the volume of the droplet can be determined. This can be true for every droplet in which reaction of at least one active reference component has occurred to a sufficient level to cause a detectable change in the level of a signal detected by the detector; this will generally be true for every droplet to be included in analysis. If the cross-section of the interrogation region is less than the average spherical cross-section of the droplets, the volume estimate becomes more accurate, as differences in droplet size or shape will not lead to differences in signal in the interrogation region because the droplet becomes elongated as it passes through the region.

It will be appreciated that useful subsample characteristics can also be inferred from one or more signals from subsamples that are not signals from reaction products of at least one active reference component or from one or more targets of interest. For example, in certain embodiments a passive reference component is used; additionally or alternatively, one or more signals produced by subsamples independent of the presence or absence of target, passive, or active reference components may be used. In both cases, the signal will be present in all subsamples, regardless of whether reaction products have formed, providing an accurate overall count of subsamples, and the signal or signals can also be used to infer subsample volume, e.g., in embodiments in which droplets flow through an interrogation region.

In certain embodiments, a first reference signal from reaction products of a first active reference component, e.g., a first reference signal from a first reference dye component that produces a first reference optical signal in the presence of the first active reference component reaction products, such as amplified first reference oligonucleotides in a PCR system, can be different from a first target signal from reaction products of a first target of interest, e.g., a first target signal from a first target dye component that produces a first target optical signal in the presence of the first target of interest reaction products, such as amplified first target nucleic acid in a PCR system. In that case, the signals can be detected in different channels of a detector and generally are independent or substantially independent of each other. In other embodiments, the signals are the same, e.g., the same dye is used for detection of both reaction products of a first active reference component and a first target of interest. In this case, the signal may be detected in a single channel of a detector, and the presence of the target of interest can be inferred from peak height, which will be greater than peak height in the absence of the target of interest. Peak heights and/or other characteristics of subsamples that do not contain the target of interest but only the active reference component can still be used to normalize target peak heights and/or other characteristics of sub samples containing the target of interest, in certain embodiments; for example, peak heights and/or other characteristics of droplets in a flowing series of droplets can be used, where peak heights and/or other characteristics of droplets containing only active reference component can be used to normalize peak heights in droplets. The use of the same detectable label for a first active reference and a first target of interest allows greater multiplexing, for example, for additional targets of interest that can have detectable labels different from the first label.

In certain embodiments, subsamples comprise components that can produce at least two different detectable signals from reaction products from one or more active reference components.

In certain embodiments, an active reference component can comprise at least two sections that produce at least two different reaction products in response to a reaction stimulus. For example, in a dPCR system, an active reference oligonucleotide can be provided that has a first nucleotide sequence that is amplified by a first set of primers and a second oligonucleotide sequence that is amplified by a second set of primers, where the amplification products of the first and second sequences are detected by first and second probes, e.g., dyes that produce optical signals. In other embodiments, separate first and second active reference components are provided, wherein the first and second active reference components are different and wherein the first and second active reference components produce different reaction products when subjected to a reaction stimulus. For example, in a dPCR system a first active reference oligonucleotide comprising a first sequence that is amplified by a first set of primers and a second active reference oligonucleotide comprising a second sequence that is amplified by a second set of primers can be provided, wherein the reaction products of the first and second sequences are detectable by first and second probes, e.g., dyes, that produce optical signals, and wherein the first and second active reference oligonucleotides and/or the first and second sets of primers are different. In certain embodiments, the first and second oligonucleotide sequences are different. In certain embodiments, the first and second primer sets are different. In certain embodiments, the first and second primer sets are the same. The first sequence and primers can have a different level or type of function than the second sequence and primers. For example, the primer set for the first sequence can be functional at low temperatures and the primer set for the second sequence can be functional at a higher temperature, or both can be functional at low temperatures but only the primer set for the second sequence is functional at high temperatures. In such embodiments, the likelihood of production of detectable reaction products is increased and thus the level of false negative for controls is decreased, since each sequence/primer set is preferentially amplifiable in a specific temperature range, to ensure that the active reference is detected no matter the reaction temperature. In addition, the ratio of the two different reaction products can give a readout as to the actual temperature experienced during the reaction, e.g., in case there is an error in set point and/or readout in the instrument performing the droplet analysis. Modification of the optimal binding temperature for, e.g., each primer set can be any suitable modification, such as sequence modification, oligonucleotide length, oligonucleotide secondary and tertiary structure modification, and/or chemical modifications to the oligonucleotides.

Modification of active reference components and/or provision of active reference components that are incapable or substantially incapable of cross-reacting with targets of interest may be used in order to maintain independence of active reference components and targets of interest. For example, in a dPCR system, an active reference oligonucleotide can contain one or more non-natural nucleotides and a primer set provided that contains complementary non-natural nucleotides that bind with the active reference non-natural nucleotides but not with natural nucleotides or substantially not with natural nucleotides; thus, when a sample is analyzed potentially containing one or more natural target nucleic acid sequences, there is no or substantially no cross-reactivity between target nucleic acid sequences and active reference sequences.

Thus, in a dPCR system, one embodiment provides a first primer set and at least two different physically distinct and sequence distinct oligonucleotide sequences that act as active reference components; the two different oligonucleotide sequences can be different sections of a single oligonucleotide or sequences on two separate oligonucleotides. Detectable labels are provided that can be the same for the different sequences or different, for example, sequence-specific detection dyes or sequence-independent detection dye.

In certain embodiments the primer set may comprise one or more modifications to enable binding specificity to only the target sequence as well as provide the thermal stability to bind at any temperature set point utilized during the analysis.

In certain embodiments, provided is a method of performing droplet-based assays in which a first signal is generated using an active reference, where the active reference is present in each of the droplets formed from a sample; for example as described in the instances below, and further signals are separately detected from the plurality of droplets. Intensity of the first signal correlating to the active reference is substantially independent of the signals correlating to target measurements. Droplet statistics and reaction performance are collected from the first signal allowing for the use of droplet statistics in the analysis of the droplet plurality, including but not limited to droplet count, acceptance/rejection, statistical correction of concentration determined in droplet analysis, as well as droplet size and volumetric determination for accurate quantification of sample volume prior to emulsification. Concentrations of the targets analyzed may be determined by any number of signals other that the first signal.

In any of these instances, the nucleic acid modifications include but are not limited to locked nucleic acid, peptide nucleic acid, and the like.

Exemplary reporters comprise at least one dye, such as a fluorescent dye or an energy transfer pair, and/or at least one oligonucleotide including but not limited to hydrolysis probes and/or an intercalating dye (e.g., EvaGreen, SYBR Green, ethidium bromide, etc.).

The reference dye may be a substance that is retained in each droplet and is not soluble in the carrier fluid used during droplet analysis. Alternatively, the reference dye may be a substance soluble in both the carrier fluid and the aqueous fluid. In some instances, the reference dye may be supplied by the carrier fluid to the aqueous fluid.

In any of these instances, a reaction is prepared to test for at least one analyte by combining the sample with a mixture of reagents for sample analysis. The sample is subsequently separated into partitions, e.g., in separate containers, or emulsified in such a manner that at least one of the droplets in the plurality is negative for the target analyzed but each droplet contains at least one copy of the active reference material. The emulsion comprises partitions of a dispersed phase in a continuous phase; typically, the dispersed phase is supplied by an intake system, e.g., as a sample or portion of a sample that is taken up by the intake system, and continuous phase is supplied, at least in part, by a process system. In certain embodiments, the intake system and the process system are separate, e.g., at no time is there continuous flow between the intake system and the process system.

The abovementioned emulsion may be generated and stored in a vessel or generated and continuously flowed through an analytical system for processing.

The emulsion may be generated using any droplet generating method including but not limited to bulk emulsification (e.g. sonication, vortexing, vigorous pipetting, shaking, blending), droplet sprayers, microfluidic generators (e.g. cross-flow, T-junctions, 3D-flow focusing generators, orifice generators). The droplets may be monodisperse or polydisperse. In preferred embodiments the droplets are monodisperse with limited volumetric variability, for example, less than 50, 40, 30, 20, 18, 15, 12, 10, 9, 8, 7, 6, 5, 4, 3, 2, or 1% volumetric variability.

The emulsion comprises partitions of a dispersed phase in a continuous phase. In some instances, the dispersed phase in an aqueous phase and the continuous phase is an oil. In further instances the continuous phase is a fluorinated oil. In further instances the continuous phase also includes a fluorosurfactant to stabilize the emulsion.

The oil generally includes any synthetic or naturally occurring liquid compound or mixture of liquid compounds that is immiscible with water. Exemplary oils include but are not limited to silicone oil, mineral oil, fluorinated oil, vegetable oils, or a combination thereof. Exemplary fluorinated oils include but are not limited to the Fluorinert and Novec series of oils by 3M, include Novec 7500.

In some instances the dispersed phase is supplied by an intake system as point of a liquid handling system, e.g., as a sample or portion of a sample that is taken up by the intake system, and continuous phase is supplied, at least in part, by the process system.

In some instances the reaction within the droplets is initiated by a chemical or physical source. This may include but are not limited to sources like thermal energy, electromagnetic energy, or chemical initiators.

For example, the reaction may be performed by heating the droplets above room temperature. In the case of PCR, the droplet plurality would be incubated at multiple different temperatures in repetition to produce the desire enzymatic function as known in the art. In some instances thermal cycling occurs after droplets are collected in vessels. In other instances, thermal cycling occurs as droplets move through an analytical system through multiple temperature zones.

Any suitable reactor may be utilized to initiate and/or propagate reactions in the droplets in flowing systems.

The process system can comprise a detector for detecting one or more characteristics of partitions as they flow through the detector. The detector can be any suitable detector, such as a detector as described herein. Thus, for example, partitions flow through the detector in single file in a conduit that includes an interrogation region where, e.g. electromagnetic radiation from the flow through the interrogation region, such as electromagnetic radiation from a partition flowing through the interrogation region, is emitted to be detected by one or more detection elements. The detector can be configured so that electromagnetic radiation from partitions that is detected by the detection element all, or substantially all, comes from individual partitions as they flow through the interrogation region; that is, there is little or no overlap in detected electromagnetic radiation from one partition to another. 1) In certain embodiments, the detector comprises an optical restriction configured and positioned between the interrogation region and the detection element so that only a portion of electromagnetic radiation from the interrogation region that could otherwise be detected by the detection element is detected, for example, less than 80, 70, 60, 50, 40, 30, 20, or 10% of the electromagnetic radiation, such as less than 1%. In certain embodiments, systems and methods include an intake system, a process system, an injector positioned between the intake system and the process system where the injector can be in fluid communication with the intake system, in fluid communication with the process system, but not both simultaneously, where the process system comprises a detector comprising an optical restriction. In certain embodiments, systems and methods include a partitioner and a detector, where the detector comprises an optical restriction. 2) In certain embodiments, the region of the conduit in the interrogation region has a cross-sectional area that is equal to or less than the average spherical cross-sectional area of partitions flowing through the detector, such as less than 90% or less than 50%. In certain embodiments, systems and methods include an intake system, a process system, an injector positioned between the intake system and the process system where the injector can be in fluid communication with the intake system, in fluid communication with the process system, but not both simultaneously, where the process system comprises a detector comprising a conduit comprising an interrogation region where the region of the conduit in the interrogation region has a cross-sectional area that is equal to or less than the average spherical cross-sectional area of partitions flowing through the detector, such as less than 95%, or 90% or less than 50%. In certain embodiments, systems and methods include a partitioner and a detector, where the detector comprising a conduit comprising an interrogation region where the region of the conduit in the interrogation region has a cross-sectional area that is equal to or less than the average spherical cross-sectional area of partitions flowing through the detector, such as less than 90% or less than 50%. 3) In certain embodiments the detector comprises an excitation source, or a plurality of excitation sources, such as at least 2, 3, 4, or 5 excitation sources, for supplying electromagnetic radiation to the interrogation region, where the excitation source or sources comprise a lock-in amplification system. In such systems only a single detection element, e.g., photodetection element, such as a silicon photomultiplier, may be used, even with a plurality of excitation sources. In certain embodiments, systems and methods include an intake system, a process system, an injector positioned between the intake system and the process system where the injector can be in fluid communication with the intake system, in fluid communication with the process system, but not both simultaneously, where the process system comprises a detector and the detector comprises an excitation source, or a plurality of excitation sources, such as at least 2, 3, 4, or 5 excitation sources, for supplying electromagnetic radiation to the interrogation region, where the excitation source or sources comprise a lock-in amplification system; in certain embodiments, only a single detection element is used. 4) In certain embodiments the detector comprises a partition separation system that separates partitions before they reach the interrogation region, e.g., by adding continuous phase between partitions before they reach the interrogation region. In certain embodiments, systems and methods include an intake system, a process system, an injector positioned between the intake system and the process system where the injector can be in fluid communication with the intake system, in fluid communication with the process system, but not both simultaneously, where the process system comprises a detector that comprises a partition separation system that separates partitions before they reach the interrogation region, e.g., by adding continuous phase between partitions before they reach the interrogation region. 5) Systems and methods provided herein may also include one or more disengager, e.g., a system that removes continuous phase from an emulsion, for example, after a partitioner but prior to a reactor, or after a detector, or both, and, in certain embodiments, adds back some or all of the removed continuous phase to the process system, e.g., at a partition separation system. In certain embodiments, systems and methods include an intake system, a process system, an injector positioned between the intake system and the process system where the injector can be in fluid communication with the intake system, in fluid communication with the process system, but not both simultaneously, where the process system comprises one or more disengager, e.g., a system that removes continuous phase from an emulsion, for example, after a partitioner but prior to a reactor, or after a detector, or both, and, in certain embodiments, adds back some or all of the removed continuous phase to the process system, e.g., at a partition separation system. 6) In certain embodiments the conduit of the interrogation region is configured to have the same or substantially the same transmittance, e.g., for electromagnetic radiation from excitation sources that reaches the interrogation region and for electromagnetic radiation from the interrogation region that is detected by the detection element, around the circumference of the conduit; for example, the conduit can be a tube, such as a tube with a circular or substantially circular cross-section. Such a configuration can allow for, e.g., coplanar or substantially coplanar arrangement of a plurality of excitation sources, such as at least 2, 3, 4, or 5 excitation sources, and/or one or more detection elements, such as in a plane orthogonal or substantially orthogonal to an axis of flow of partitions in the interrogation region. In certain embodiments, systems and methods include an intake system, a process system, an injector positioned between the intake system and the process system where the injector can be in fluid communication with the intake system, in fluid communication with the process system, but not both simultaneously, where the process system comprises a detector comprising an interrogation region comprising a conduit, where the conduit of the interrogation region is configured to have the same or substantially the same transmittance, e.g., for electromagnetic radiation from excitation sources that reaches the interrogation region and for electromagnetic radiation from the interrogation region that is detected by the detection element, around the circumference of the conduit; for example, the conduit can be a tube, such as a tube with a circular or substantially circular cross-section. In certain embodiments, systems and methods provided herein include a detector with characteristics of at least one of 1), 2), 3), 4), 5) and 6). In certain embodiments, systems and methods provided herein include a detector with characteristics of at least two of 1), 2), 3), 4), 5) and 6). In certain embodiments, systems and methods provided herein include a detector with characteristics of at least three of 1), 2), 3), 4), 5) and 6). In certain embodiments, systems and methods provided herein include a detector with characteristics of at least four of 1), 2), 3), 4), 5) and 6). In certain embodiments, systems and methods provided herein include characteristics of at least five of 1), 2), 3), 4), 5) and 6). In certain embodiments, systems and methods provided herein include a detector with characteristics of all of 1), 2), 3), 4), 5) and 6). In certain embodiments, systems and methods provided herein include an intake system and a process system, wherein the intake system and the process system are never in fluid communication, and the process system comprises a detector with characteristics of at least one of 1), 2), 3), 4), 5) and 6). In certain embodiments, systems and methods provided herein include an intake system and a process system, wherein the intake system and the process system are never in fluid communication, and the process system comprises a detector with characteristics of at least two of 1), 2), 3), 4), 5) and 6). In certain embodiments, systems and methods provided herein include an intake system and a process system, wherein the intake system and the process system are never in fluid communication, and the process system comprises a detector with characteristics of at least three of 1), 2), 3), 4), 5) and 6). In certain embodiments, systems and methods provided herein include an intake system and a process system, wherein the intake system and the process system are never in fluid communication, and the process system comprises a detector with characteristics of at least four of 1), 2), 3), 4), 5) and 6). In certain embodiments, systems and methods provided herein include an intake system and a process system, wherein the intake system and the process system are never in fluid communication, and the process system comprises a detector with characteristics of at least five of 1), 2), 3), 4), 5) and 6). In certain embodiments, systems and methods provided herein include an intake system and a process system, wherein the intake system and the process system are never in fluid communication, and the process system comprises a detector with characteristics of all of 1), 2), 3), 4), 5) and 6).

Signal generated by the droplets may be detected from the droplet plurality as droplets pass from the reactor to the detector system. The signal may be detected from any number of droplets in the plurality. The signal from the droplets may be detected in multitude and the signal for each droplet deconvoluted afterwards or the signal may be detected in singularity. The signal may be collected once or many datapoints per droplet may be collected and integrated together. Signal may be detected as droplets are stationary, moving in a floating system, or moving is a fixed contained. In a preferred embodiment, signals from the droplet plurality are collected for each droplet individually as the pass through a signal interrogation region in the detector assembly. In this embodiment, signal is collected at a predetermined rate based on the hardware and each droplet receives a certain number of signal values based on the sampling rate and the velocity of the droplet as it travels through the interrogation region. At least 1 data point is collected per droplet. In certain embodiments, an average of at least 1, 2, 3, 4, 5, 6, 7, 8, 9, 10, 12, 15, 20, 25, 30, 40, 50, 60, 70, 80, 90, 100, 120, 150, 200, 300, or 400 data points, and/or not more than 2, 3, 4, 5, 6, 7, 8, 9, 10, 12, 15, 20, 25, 30, 40, 50, 60, 70, 80, 90, 100, 120, 150, 200, 300, 400, or 500 data points are collected per droplet, e.g., an average of 1-500, or 1-200, or 1-100, or 2-500, or 2-200, or 2-100, or 5-500, or 5-200, or 5-100, or 5-50 data points are collected per droplet. In certain embodiments an average of between 5 and 50 data points are collected per droplet.

In the case of moving droplets, information about the droplets may be generated using the collected signals from both the active reference channel as well as one or more channels utilized for target analysis. Signal collected continuously by the detector may be analyzed with respect to time. This data typically resembles peaks, pulses, or spikes due to signal rising from baseline as the droplet passes through the detection region, peaking to a maximum value as the droplet enters the center of the interrogation region, and then falling back to baseline as the droplet leaves the detection region. The major characteristics (such as height, width, shape) of these peaks have utility in the interpretation of the contents and quality of the droplets being analyzed.

Using this data, droplet positive for the target may be counted using the one or more channels dedicated to target analysis. Total droplets may be counted utilizing the active reference channel. Information about droplet quality may be collected using the active reference channel. These quality statistics include droplet count, width, area, and the like.

The concentration of the one or more targets being analyzed in the original sample may be determined using the count of target positive droplets and the total number of droplets in the plurality. Droplet counts and therefore target concentration may be adjusted using appropriate statistical methods including but not limited to Poisson statistics.

Droplet volume may be calculated using the total number of droplets and their width values as long as the droplet velocity and channel dimensions are known. This allows for statistical correction of sample volume for more accurate measurements. Additionally, droplets of poor quality may be disregarded from the analysis and the analyzed volume corrected as needed.

Volume may also be preset in the analytical instrument through calibration by the instrument manufacturer rather than determined through volumetric counting of droplets.

Volume may be cross-correlated between the two measurement methods for higher precision.

When utilizing Poisson statistics for statistical correction, but the total number of droplets and the number of positive droplets must be known. A correction factor may be applied to the droplets to account for random loading of target within the droplet plurality. This correction factor may be calculated using the equation C_{corr}=Cₜₒₜₐₗ*Cₜₒₜₐₗ/C_{neg}) where C_{corr} is the Poisson corrected count of positive droplets, Cₜₒₜₐₗ is the total count of droplets analyzed in the plurality and C_{neg} is the count of negative droplets as determined by C_{neg}=Cₜₒₜₐₗ−Cₚₒₛ, where Cₚₒₛ is the count of target positive droplets. This requires at least one droplet in the plurality to not be target positive.

Target concentration may be calculated with or without applying statistical correction. By dividing the target positive droplet count (with or without correction) by the sample volume determined by either of the abovementioned means.

The assay required for the active reference material might exist as distinct units added to the reaction during set up or may be provided incorporated into the mixture before setup by a supplier.

### Compositions

In certain embodiments, provided herein is a composition comprising a first plurality of subsamples created from a first sample, where all or substantially all of the first sample is divided into the plurality of subsamples, and where a portion of the subsamples contain a target of interest, a portion of the subsamples do not contain the target of interest, and each of the subsamples contains a first active reference component. A sample from which the subsamples are derived may be a sample that is introduced into a digital process system, for example an aliquot of a larger sample that is sampled by the system for processing. At least 50, 60, 70, 80, 90, 95, or 99% of the first sample can be divided into the subsamples. Each of the subsamples can further comprise a second active reference component, different from the first active reference component. Each of the subsamples can further comprise a passive reference component. In certain embodiments, the first target of interest, the first active reference component, and the second active reference component, if present, are oligonucleotides. In certain of these embodiments, each subsample further includes a first target primer set for the first target of interest, a first active reference primer set, different from the first target primer set, for the first active reference oligonucleotide, and, if a second active reference oligonucleotide is present, a second active reference primer set for the second active reference oligonucleotide. In certain embodiments each subsample comprises a first and second active reference oligonucleotide and first and second active reference primer sets, wherein the first active reference oligonucleotide and the first active reference primer set has a first optimum temperature for amplification and the second active reference oligonucleotide and the second active reference primer set has a second optimum temperature for amplification, different from the first optimum temperature. The subsamples can be droplets of dispersed phase in a continuous phase.

In certain embodiments, provided herein is a composition comprising at least two different oligonucleotides and one or more primers for amplifying the oligonucleotides by PCR, where the oligonucleotides and the primers comprise modified nucleotides and the primers will not amplify natural oligonucleotides. In certain embodiments, the two different oligonucleotides and their respective primer sets have different optimum temperatures for PCR. In certain embodiments the composition also comprises a target oligonucleotide of interest, where the target oligonucleotide is composed only of natural nucleotides; the composition may further include primers for amplification of the target oligonucleotide by PCR.

### Kits

In certain embodiments provided herein is a kit for quality control of a digital process, wherein the kit includes a first container comprising an active reference component for the digital process, and a second container comprising reagents necessary and/or useful in reaction of the first active reference component in the digital process to produce a detectable change in the first active reference component. The first and second containers may be the same or different. The kit may also include a third container comprising a second active reference component for the digital process, different from the first, and a fourth container comprising reagents necessary and/or useful in reaction of the second active reference component in the digital process to produce a detectable change in the second active reference component. The third and fourth containers may be the same or different; the first, second, third, and fourth containers may also be the same or different. The kit may further include a passive reference for the digital process in a fifth container, where the fifth container may be the same or different from the first, second, third, and/or fourth containers. The kit may further include instructions for use, further reagents necessary and/or useful in the digital process, and/or packaging to contain some or all of the components of the kit. The kit may provide all necessary components for quality control of the digital process in such form that they are useable by a consumer with little or no modification, e.g., no modification or simple modification such as dilution or addition of readily available reagents or the like. In certain embodiments the kit provides all necessary components in a form that requires no modification for use by the consumer. In certain embodiments the digital process is a digital PCR process and the first active reference component and second active reference component, if present, are first and second active reference oligonucleotides, as described herein, and the reagents necessary and/or useful for reaction include a first set of primers for amplification the first active reference oligonucleotide and, if a second active reference oligonucleotide is used, a second set of primers for amplification of the second active reference oligonucleotide, where the first and second set of primers may be the same or different. Reagents may further include, e.g., a PCR Mastermix, fluorescent probes for detection of amplification products of the first active reference oligonucleotide and second active reference oligonucleotide, if present. The kit may also comprise a passive reference component, e.g., a dye that is not involved in the PCR process.

### EMBODIMENTS

In embodiment 1 provided herein is a method of quality control in a digital process comprising (i) dividing a sample into a plurality of subsamples, wherein (a) a portion of the subsamples potentially comprise a target of interest; (b) a portion of the subsamples do not comprise the target of interest; (c) each of the subsamples comprises an average of at least one of a first active reference component; (ii) exposing the subsamples to a stimulus that causes a first detectable change in the target of interest, if present, and that also causes a second detectable change in the first active reference component; and (iii) detecting the first change in the target of interest, if present, and the second change in the first active reference component, in individual subsamples.

In embodiment 2 provided herein is the method of claim 1 wherein the plurality of subsamples are in separate containers.

In embodiment 3 provided herein is the method of claim 1 or 2 wherein the plurality of subsamples are partitions of dispersed phase comprising the target of interest, if present, and the first active reference component, in a continuous phase.

In embodiment 4 provided herein is the method of claim 3 wherein the partitions are stationary.

In embodiment 5 provided herein is the method of claim 3 wherein the partitions and continuous phase are flowing.

In embodiment 6 provided herein is the method of any one of claims 3 through 5 wherein the partitions are polydisperse.

In embodiment 7 provided herein is the method of any one of claims 3 through 5 wherein the partitions are polydisperse.

In embodiment 8 provided herein is the method of any previous claim wherein the first detectable change causes a first detectable signal and the second detectable change causes a second detectable signal.

In embodiment 9 provided herein is the method of claim 8 wherein the first and second detectable signals are the same signal.

In embodiment 10 provided herein is the method of any previous claim wherein each of the subsamples comprises an average of at least one of a second active reference component and the method comprises exposing the subsamples to the stimulus to cause a third detectable change in the second active reference component, and detecting the third detectable change in the second active reference component.

In embodiment 11 provided herein is the method of claim 10 wherein the first and second active reference components are separate.

In embodiment 12 provided herein is the method of claim 10 wherein the first and second active reference components are separate parts of a single component.

In embodiment 13 provided herein is the method of any previous claim wherein each of the subsamples comprises a passive reference component and detecting the passive reference component in each of the subsamples.

In embodiment 14 provided herein is the method of any previous claim wherein the target of interest and the first active reference component are oligonucleotides and the stimulus causes a polymerase chain reaction in the target of interest and the first active reference component.

In embodiment 15 provided herein is the method of any one of claims 10 through 14 wherein the target of interest and the first and second reference components are oligonucleotides and the stimulus causes a polymerase chain reaction in the target of interest and in the first and second oligonucleotides.

In embodiment 16 provided herein is the method of claim 15 wherein the first and second reference oligonucleotides use the same pair of primers.

In embodiment 17 provided herein is the method of claim 15 wherein the first and second reference oligonucleotides use the different pairs of primers.

In embodiment 18 provided herein is the method of claim 16 or 17 wherein the first and second reference oligonucleotides and their primers have different optimal reaction temperatures.

In embodiment 19 provided herein is a method of performing quality control of digital polymerase chain reaction (PCR) in droplets comprising (i) forming an emulsion of a plurality of droplets comprising dispersed phase in a continuous phase, wherein (a) a portion of the plurality of droplets potentially comprises a first oligonucleotide target of interest, (b) a portion of the plurality of droplets does not comprise the first oligonucleotide target of interest, and (c) each of the plurality of droplets comprises a first oligonucleotide active reference component and a second oligonucleotide active reference component, different from the first; (ii) performing PCR on the droplets so that (a) the first oligonucleotide target of interest, if present, is amplified and provides a first detectable target signal, (b) the first oligonucleotide active reference component is amplified and provides a first detectable reference signal, and (c) the second oligonucleotide active reference component is amplified and provides a second detectable reference signal; and (iii) in individual droplets, detecting the first detectable target signal, if present, and detecting the first and second detectable reference signals.

In embodiment 20 provided herein is a composition comprising a first plurality of subsamples created from a first sample, wherein all or substantially all of the first sample is divided into the plurality of subsamples, and wherein (i) a portion of the subsamples comprise a first target of interest; (ii) a portion of the subsamples does not comprise the first target of interest; and (iii) each of the subsamples comprises a first active reference component.

In embodiment 21 provided herein is the composition of claim 20 wherein each of the subsamples further comprises a second active reference component, different from the first active reference component.

In embodiment 22 provided herein is the composition of claim 20 or 21 wherein each of the subsamples further comprises a passive reference component.

In embodiment 23 provided herein is the composition of any of claims 20 through 22 wherein the first target of interest, the first active reference component, and the second active reference component, if present, are oligonucleotides.

In embodiment 24 provided herein is the composition of claim 23 wherein each subsample further includes a first target primer set for the first target of interest, a first active reference primer set, different from the first target primer set, for the first active reference, and, if a second active reference component is present, a second active reference primer set for the second active reference.

In embodiment 25 provided herein is the composition of claim 23 wherein each subsample comprises a first and second active reference oligonucleotide and first and second active reference primer sets, wherein the first active reference oligonucleotide and the first active reference primer set has a first optimum temperature for amplification and the second active reference oligonucleotide and the second active reference primer set has a second optimum temperature for amplification, different from the first optimum temperature.

In embodiment 26 provided herein is a kit for performing quality control on a digital polymerase chain reaction (dPCR) comprising (i) a first container comprising a first active reference oligonucleotide; (ii) a second container comprising a first set of primers specific for amplifying the first active reference oligonucleotide; (iii) a third container comprising a second active reference oligonucleotide, different from the first active reference nucleotide; and (iv) a fourth container comprising a second set of primers specific for amplifying the second active reference oligonucleotide.

In embodiment 27 provided herein is the kit of claim 26 wherein the first and second containers are the same container.

In embodiment 28 provided herein is the kit of claim 26 wherein the first and second containers are different containers.

In embodiment 29 provided herein is a composition comprising a plurality of droplets of dispersed phase in a continuous phase, wherein all or substantially all of the droplets are formed from a single sample, wherein (i) a portion of the plurality of droplets potentially comprises a first target oligonucleotide; (ii) a portion of the plurality of droplets does not comprise the first target oligonucleotide; and (iii) each of the droplets of the plurality of droplets comprise a first active reference oligonucleotide.

In embodiment 30 provided herein is a composition comprising at least two different oligonucleotides and one or more primers for amplifying the oligonucleotides by PCR, wherein the oligonucleotides and the primers comprising modified nucleotides that are not natural and not found in natural samples, and the primers will not amplify natural oligonucleotides.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of quality control in a digital process comprising
(i) dividing a sample into a plurality of subsamples, wherein
(a) a portion of the subsamples potentially comprise a target of interest;
(b) a portion of the subsamples do not comprise the target of interest;
(c) each of the subsamples comprises an average of at least one of a first active reference component;
(ii) exposing the subsamples to a stimulus that causes a first detectable change in the target of interest, if present, and that also causes a second detectable change in the first active reference component; and
(iii) detecting the first change in the target of interest, if present, and the second change in the first active reference component, in individual subsamples.

2. The method of claim 1 wherein the plurality of subsamples are partitions of dispersed phase comprising the target of interest, if present, and the first active reference component, in a continuous phase.

3. The method of claim 2 wherein the partitions are stationary.

4. The method of claim 2 wherein the partitions and continuous phase are flowing.

5. The method of claim 1 wherein the first detectable change causes a first detectable signal and the second detectable change causes a second detectable signal.

6. The method of claim 5 wherein the first and second detectable signals are the same signal.

7. The method of claim 1 wherein each of the subsamples comprises an average of at least one of a second active reference component and the method comprises exposing the subsamples to the stimulus to cause a third detectable change in the second active reference component, and detecting the third detectable change in the second active reference component.

8. The method of claim 7 wherein the first and second active reference components are separate.

9. The method of claim 7 wherein the first and second active reference components are separate parts of a single component.

10. The method of claim 1 wherein each of the subsamples comprises a passive reference component and detecting the passive reference component in each of the subsamples.

11. The method of claim 1 wherein the target of interest and the first active reference component are oligonucleotides and the stimulus causes a polymerase chain reaction in the target of interest and the first active reference component.

12. The method of claim 7 wherein the target of interest and the first and second reference components are oligonucleotides and the stimulus causes a polymerase chain reaction in the target of interest and in the first and second oligonucleotides.

13. The method of claim 12 wherein the first and second reference oligonucleotides use a different pair of primers.

14. The method of claim 13 wherein the first and second reference oligonucleotides and their primers have different optimal reaction temperatures.

15. A composition comprising a first plurality of subsamples created from a first sample, wherein all or substantially all of the first sample is divided into the plurality of subsamples, and wherein
(i) a portion of the subsamples comprise a first target of interest;
(ii) a portion of the subsamples does not comprise the first target of interest; and
(iii) each of the subsamples comprises a first active reference component.

16. The composition of claim 15 wherein each of the subsamples further comprises a second active reference component, different from the first active reference component.

17. The composition of claim 15 wherein each of the subsamples further comprises a passive reference component.

18. The composition of claim 15 wherein the first target of interest, the first active reference component, and the second active reference component, if present, are oligonucleotides.

19. The composition of claim 18 wherein each subsample further includes a first target primer set for the first target of interest, a first active reference primer set, different from the first target primer set, for the first active reference, and, if a second active reference component is present, a second active reference primer set for the second active reference.

20. A kit for performing quality control on a digital polymerase chain reaction (dPCR) comprising
(i) a first container comprising a first active reference oligonucleotide;
(ii) a second container comprising a first set of primers specific for amplifying the first active reference oligonucleotide;
(iii) a third container comprising a second active reference oligonucleotide, different from the first active reference nucleotide; and
(iv) a fourth container comprising a second set of primers specific for amplifying the second active reference oligonucleotide.
